(19) United States
(12) Patent Application Publication
(10) Pub. No.: US 2017/0202880 A1
(43) Pub. Date: Jul. 20, 2017

(54) METHOD FOR PREPARING NOBLE METAL NANOPARTICLES FROM MYRRH

(71) Applicant: KING SAUD UNIVERSITY, RIYADH (SA)

(72) Inventors: EBTESAM MOHAMMED AL OLAYAN, RIYADH (SA); MANAL AHMED GASMELSEED AWAD, RIYADH (SA); MUZZAMMIL IQBAL SIDDIQUI, RIYADH (SA); NADA MOHAMMED MERGHANI, RIYADH (SA)

(21) Appl. No.: 15/475,114

(22) Filed: Mar. 30, 2017

Related U.S. Application Data
(63) Continuation-in-part of application No. 14/884,717, filed on Oct. 15, 2015, now abandoned.

(51) Int. Cl. A61K 33/38; B22F 1/00; B22F 9/24; A61K 9/00; A61K 9/14

(52) U.S. Cl. CPC A61K 33/38; A61K 9/0014; A61K 9/148; B22F 9/24; B22F 1/0018; B22F 2009/245; B22F 2301/255; B22F 2304/054; B22F 2998/10

(57) ABSTRACT

The method for preparing metal nanoparticles includes preparing an extract of myrrh and mixing the extract with an aqueous solution including a metal salt. The mixture changes in color from light yellow to dark brown upon formation of nanoparticles. The extract of myrrh can be a water extract prepared by, for example, soaking a quantity of myrrh in water, filtering the soaked myrrh to obtain a filtered product, and then centrifuging the filtered product. The metal salt can be, for example, silver nitrate (AgNO₃). The metal nanoparticles can be spherical, spheroidal, elongated spherical, rod, and/or faceted. The metal nanoparticles can be used to treat Leishmaniasis (lesions) caused by Leishmania major.

### CROSS REFERENCE TO RELATED APPLICATIONS

The present application is a continuation-in-part of U.S. patent application Ser. No. 14/884,717, filed Oct. 15, 2015.

### BACKGROUND OF THE INVENTION

1. Field of the Invention

The present invention relates to bio-nanotechnology and particularly to a method of preparing nanoparticles synthesized from plant gum extract, which can be used for the treatment of Leishmaniasis.

2. Description of the Related Art

Nanoparticles exhibit completely new or improved properties compared to their corresponding bulk materials. Because of their size, catalytic property, ability to deliver drug, increased efficacy, and decreased toxicity, nanotechnology finds applications in various fields including healthcare, defense and day-to-day life.

Nanoparticles are generally obtained from metal salt solutions in the presence of a reducing agent and a stabilizing agent, which serves to prevent nanoparticles from aggregating. Because the nanoparticles possess a very high surface to volume ratio, they are particularly useful in applications where high surface areas are critical for success. Nanoparticles can be synthesized from chemical or natural products. Nanoparticle synthesis from natural products is usually more preferable. When compared to nanoparticles manufactured from chemicals, for example, nanoparticles made from natural products are more eco-friendly, readily available, cost effective, and have little if any side effects.

Thus, a method of producing metal nanoparticles utilizing Myrrh thereby solving the aforementioned problems is desired.

### SUMMARY OF THE INVENTION

The method for preparing metal nanoparticles includes preparing an extract of myrrh and mixing the extract with an aqueous solution including a metal salt. The mixture changes in color from light yellow to dark brown upon formation of nanoparticles. The extract of myrrh can be a water extract prepared by, for example, soaking a quantity of myrrh in water, filtering the soaked myrrh to obtain a filtered extract, and then centrifuging the filtered extract. The metal salt can be, for example, silver nitrate (AgNO₃). The metal nanoparticles can be spherical, spheroidal, elongated spherical, rod-shaped, and/or faceted. The metal nanoparticles can be used to treat leishmaniasis caused by Leishmania major.

These and other features of the present invention will become readily apparent upon further review of the following specification and drawings.

### BRIEF DESCRIPTION OF THE DRAWINGS

FIG. 1 shows a graph of surface plasmon resonance of the silver nanoparticles as observed by UV-Vis spectrum.

FIG. 2 shows a graph of the average size of the silver nanoparticles as measured by the zeta sizer.

FIG. 3A shows the transmission electron microscopy (TEM) images of the silver nanoparticles produced by the present method.

FIG. 3B shows the transmission electron microscopy (TEM) images of the silver nanoparticles produced by the present method

FIG. 4 shows the graph of elemental analyses by Energy-dispersive X-ray spectroscopy (EDX) of the silver nanoparticles.

FIG. 5 is a graph showing results of an MTT assay of cytotoxic effects on promastigotes of Leishmania major.

FIG. 6 is a graph showing the length and width of the lesion in (mm) in the two treatment groups of silver nanoparticles and Pentostam.

Similar reference characters denote corresponding features consistently throughout the attached drawings.

### DETAILED DESCRIPTION OF THE PREFERRED EMBODIMENTS

A method for preparing metal nanoparticles from the gum extract of the Commiphora molmol plant, referred to herein as “myrrh” is provided. The nanoparticles can be used in treating wounds caused by Leishmaniasis. The method of preparing metal nanoparticles from myrrh can include preparing an extract of myrrh, providing an aqueous solution including a metal salt, and mixing the myrrh extract with the aqueous solution of the metal salt solution to form metal nanoparticles in an aqueous myrrh suspension. Preferably, the myrrh extract and the aqueous solution of the metal salt are mixed for about 15 to 20 minutes at a temperature of 50° C. to 100° C. to produce the metal nanoparticles. The aqueous solution is colorless at first. When the myrrh extract is added to the aqueous solution, a color change is observable from colorless to light yellow, and from light yellow to dark brown upon formation of the metal nanoparticles.

The extract of myrrh can be a water extract prepared by, for example, soaking a quantity of myrrh in water, filtering the soaked myrrh to obtain a filtered product, and then centrifuging the filtered extract. The metal salt can be silver nitrate (AgNO₃) and the metal nanoparticles formed by the present method can be silver nanoparticles (referred to herein as myrrh silver nanoparticles). The metal nanoparticles can be spherical, spheroidal, elongated spherical, rod-shaped, and/or faceted-shape.

The metal nanoparticles can be used to treat and/or cure a zoonotic disease. For example, the metal nanoparticles can be used to treat Leishmaniasis caused by Leishmania major. A method of treating epidermal lesions caused by Leishmaniasis can include topically applying an effective amount of the myrrh silver nanoparticles and the aqueous myrrh suspension in which the myrrh silver nanoparticles are formed. The silver nanoparticles can have a mean size of about 5 nm to about 50 nm. The concentration of the myrrh silver nanoparticles applied topically can be about 10⁻³ to 10⁻⁵ moles/L.

Around 12 million people in the world are affected by Leishmaniasis. Leishmaniasis is caused by 20 different species and it is presented in three forms, cutaneous, mucocutaneous, and visceral. Conventional medicines for treating Leishmaniasis are associated with a very long healing process along with other challenges, like availability, cost, side effects, toxicity, drug resistance, low efficacy, and low immunological activity in case of vaccines. Nanoparticles can be used to treat various vector borne diseases, including Leishmaniasis.

As used herein, the term “Nanoparticle” refers to a particle having at least one dimension and sized between 1 and 100 nanometers. The metal nanoparticles can include gold or silver nanoparticles. In some embodiments, the nanoparticles disclosed herein are from about 5 nm to about 500 nm in diameter. As used herein, the term “topical administration” refers to the application of the nanoparticles with or without a pharmaceutically acceptable carrier to the external surface of the skin. Topical administration includes application of the composition to intact or broken skin and raw or open wounds. Topical administration of a pharmaceutical agent can result in a limited distribution of the agent to the skin and surrounding tissues or, when the agent is removed from the treatment area by the bloodstream, can result in systemic distribution of the agent.

As used herein, “zoonotic disease” refers to a disease that can be transmitted from animals to people or, more specifically, a disease that normally exists in animals but that can infect humans, such as Leishmaniasis. Leishmaniasis is caused by protozoan parasites from more than 20 Leishmania species that are transmitted to humans by the bites of infected female phlebotomine sandflies. There are three main forms of the disease: cutaneous, visceral and mucocutaneous: cutaneous leishmaniasis, visceral leishmaniasis or kala-azar, and mucocutaneous leishmaniasis. Commiphora molmol (Myrrh) is one species of the resin bearing plants, which grows across the Red Sea and Arabia. Myrrh plant is increasingly being recognized for various medicinal purposes.

Although nanoparticles can have anti-leishmanial effects, the present inventors have found that there is a significant increase in the anti-leishmanial effects of metal nanoparticles, e.g., gold or silver nanoparticles, that are synthesized using myrrh, as described herein. Myrrh is a resin that comes from trees belonging to the genus Commiphora—which grow in Northern Africa and the Middle East.

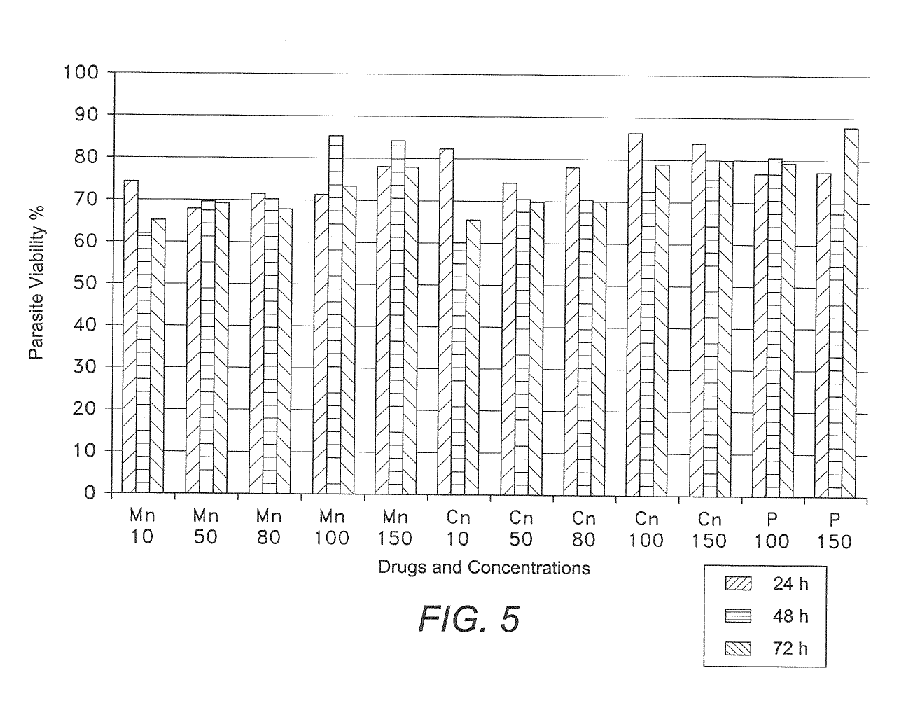

The metal nanoparticles synthesized according to the present teachings can be topically administered to a patient to treat Leishmaniasis. The metal nanoparticles synthesized according to the present teachings were shown to heal wounds, e.g., subcutaneous epidermal lesions, caused by Leishmania major within 21 days of topical application, when the nanoparticles were applied topically or superficially every day for 21 days. Parasite viability was tested by MTT (3-(4-,5-dimethylthiazol-2-yl)-2, 5-diphenyltetrazolium bromide) assay in which the metal nanoparticles synthesized from myrrh as described herein showed less viable parasites than the commercially used drug (Pentostam) used to treat Leishmania. FIG. 5 is a graph showing results of an MTT assay (described in detail in the Examples below) of cytotoxic effects of the commercial drug (Pentostam), ordinary silver nanoparticles, and silver nanoparticles synthesized from myrrh extract as described herein, on pro-mastigotes of Leishmania major. As shown in FIG. 5, the anti-Parasitic or Parasitic vaccines test of silver nanoparticles synthesized according to the present teachings showed a significant inhibition against Leishmania in in vitro trials. In addition, use of the silver nanoparticles produced according the present teachings unexpectedly reduced parasite viability to a greater extent than the commercially available silver nanoparticles. Also, unexpectedly, parasite viability was reduced using silver nanoparticles produced in accordance with the present teachings to a greater extent than that achieved using the commercially available Pentostam drug.

The present methods for synthesizing metal nanoparticles using myrrh provide a “green” or safe and environmentally benign method for producing metal nanoparticles. In addition, the Commiphora myrrha or Commiphora molmol plant is readily available, cost effective and easy to manufacture, which also provides commercial advantages.

The following examples will further illustrate the synthetic processes of making the metal nanoparticles and Leishmania major inhibition assay.

### Example 1

### Green Synthesis of Silver Nanoparticles

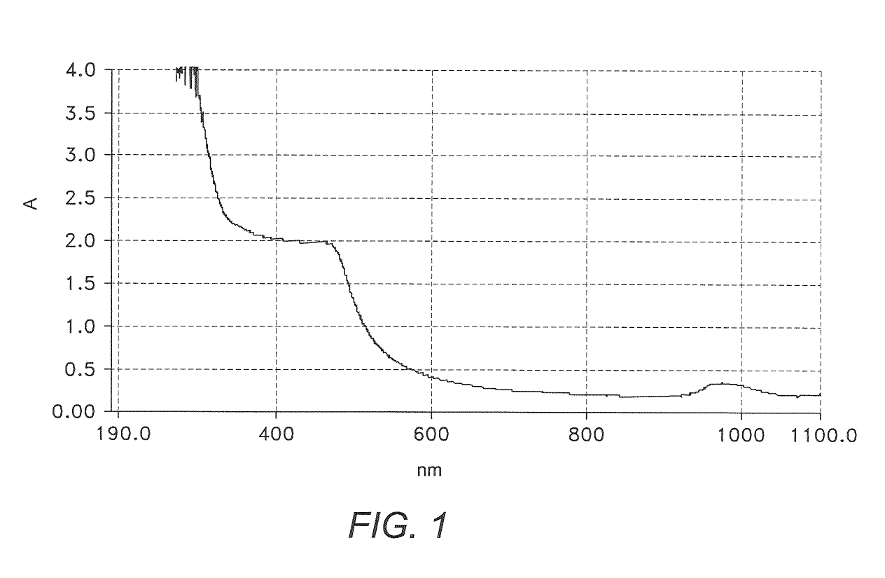

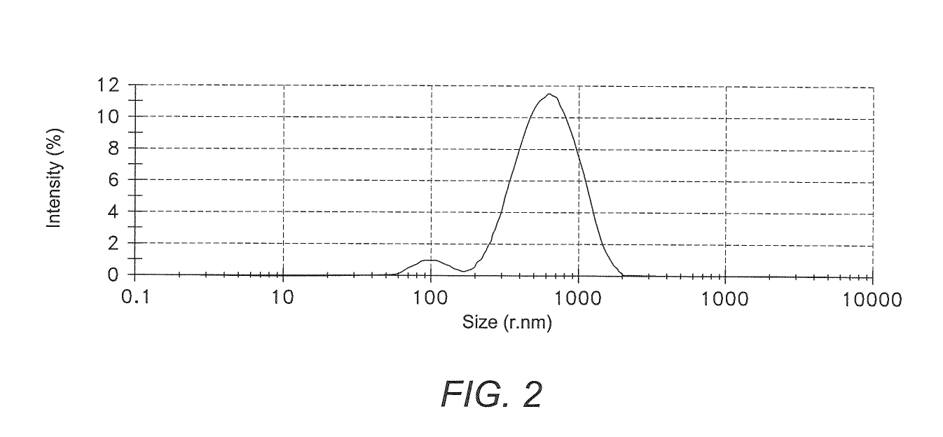

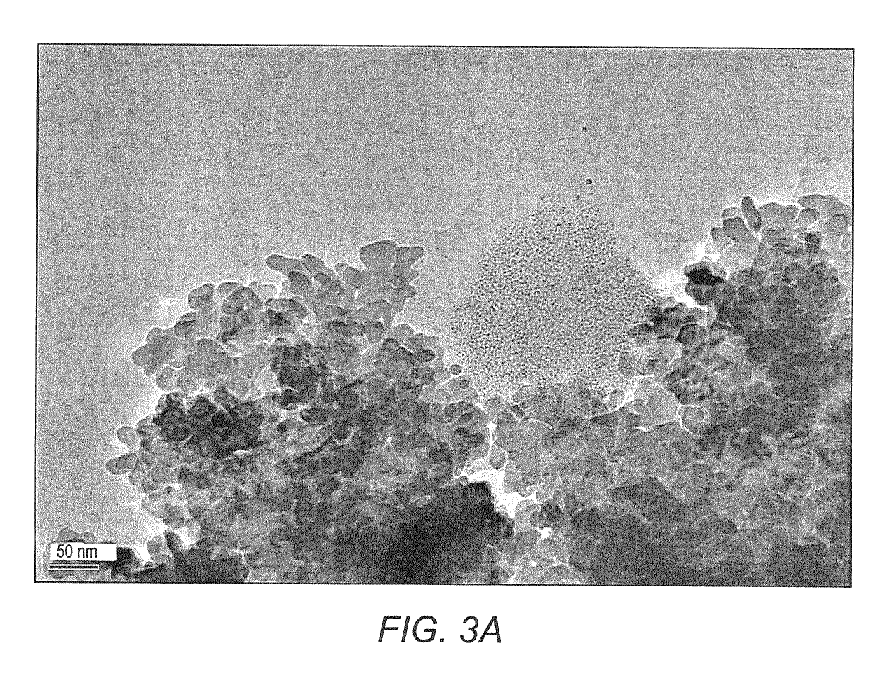

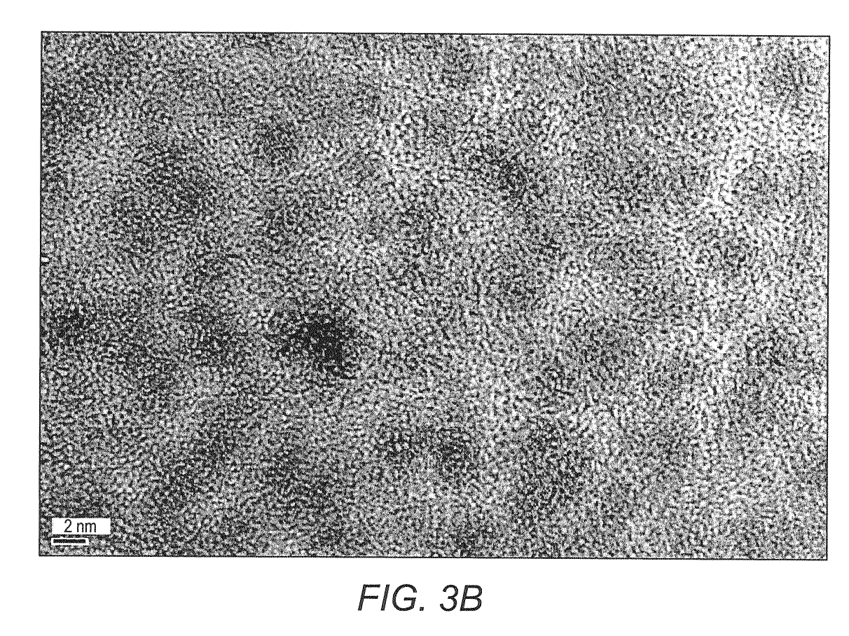

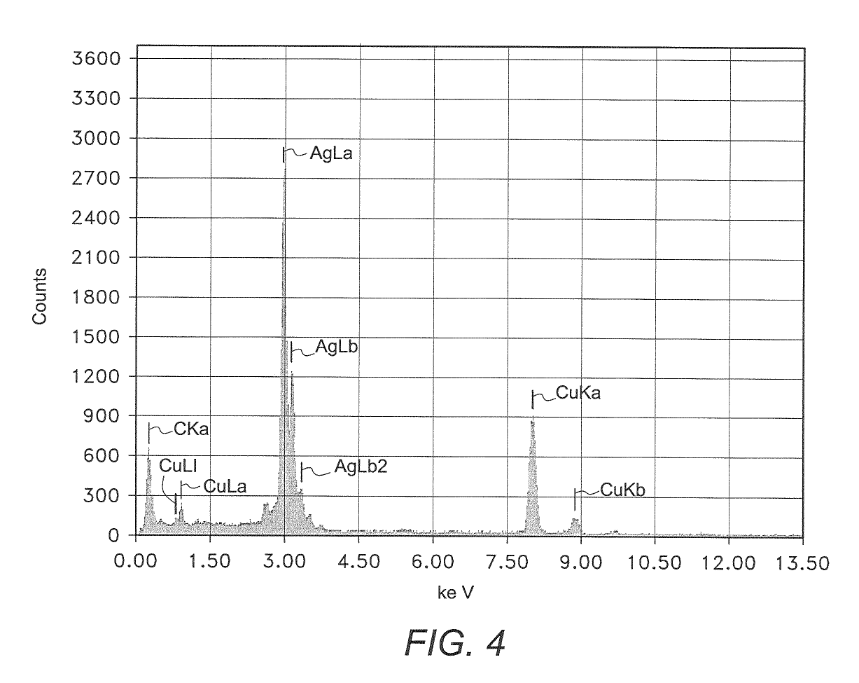

About 10 g of the gum of Commiphora molmol (myrrh) plant was soaked in about 500 ml distilled water over night, after which it was filtered and then centrifuged to prepare the extract. About 5 ml of the myrrh gum extract was added to about 50 ml of aqueous solution of 0.0009 mole/L silver nitrate (AgNO₃) and stirred for about 15 minutes at 65° C. Silver nanoparticles were formed in the aqueous myrrh suspension. The formation of nanoparticles was monitored by the color change from colorless to light-yellow and then to dark brown. The silver nanoparticles had a mean size of about 5 min to about 50 nm. FIG. 1 shows the UV-Vis spectrum of the formed silver nanoparticles. FIG. 2 shows the particle size distribution by intensity measurement using a Zetasizer. FIGS. 3A and 3B show the transmission electron microscopy (TEM) images of the silver nanoparticles produced by the present method. FIG. 4 shows the elemental analyses by Energy-dispersive X-ray spectroscopy (EDX) of the silver nanoparticles, confirming the presence of elemental silver.

### Example 2

### Parasite Viability Assay (MTT)

Anti-parasitic or parasitic vaccines were tested using silver nanoparticles synthesized by the gum extract of the plant Commiphora myrrha (myrrh) in accordance with the method described in Example 1. The MTT assay is a colorimetric assay for assessing cell viability. NAD(P)H-dependent cellular oxidoreductase enzymes may, under defined conditions, reflect the number of viable cells present. These enzymes are capable of reducing the tetrazolium dye MTT 3-(4, 5-dimethylthiazol-2-yl)-2, 5-diphenyltetrazolium bromide to its insoluble formazan, which has a purple color. Tetrazolium dye assays can also be used to measure cytotoxicity (loss of viable cells) or cytostatic activity (shift from proliferative to resting status) of potential medicinal agents and toxic materials. MTT assays are usually done in the dark since the MTT reagent is sensitive to light.

The zoonotic parasite, Leishmania major were cultured in 96 wells plate at density 2×10⁵ parasite/well in 100 μl optimized medium. The parasite was allowed to culture for 24 hours before treatment with individual concentrations of a commercial drug, ordinary silver nanoparticles, and silver nanoparticles synthesized from Myrrh extract in accordance with the present methods, i.e., 10, 50, 80, 100 and 150 mM. Treated parasites were allowed to grow further for 24 hours, 48 hours and 72 hours. At the end of the incubation period and concentration point, 100 μof 0.22 μm filter-sterilized of 3-(4,5-dimethylthiazol-2-yl)-2,5-diphenyltetrazolium bromide (MTT, Sigma Aldrich, UK) was added at 26° C. at final concentration of 5 mg/ml. The 96 wells plate was kept in the dark for 3 hours before the medium containing MTT was removed. Then 100 μl of Dimethyl sulfoxide (DMSO) was added to dissolve formazan crystals. The 96-well-plate was also shaken for 15 minutes in the dark to help dissolve the formazan crystals. The optical density (O.D.) of each treatment was measured at 570 nm using Lab systems Multiskan EX Version 3.0 (Helsinki, Finland). Each experiment was performed in three replicates. Values of optical densities were normalized according to the control (untreated parasites). Therefore, parasite viability values of untreated parasites should be 100% while values of treated parasites have values below or above 100%. The following equation (1) was used for calculations:

$$Parasite Viability ( \% ) = ( \frac{Absorbance of individual treatment}{Absorbance of the control} ) \times 100 \qquad ( 1 )$$

Table 1 shows the parasite viability of Leishmania major in different concentrations of Mn (Myrrah/silver nanoparticles produced by the present methods), Cn (Silver nanoparticles produced from chemical materials only in accordance with conventional methods), and P (Pentostam drug).

TABLE 1

| | Drugs and | Incubation | Incubation | Incubation |
|---|---|---|---|---|
| | Concentrations | Time | Time | Time |
| S. No. | (mM) | (24 hrs.) | (48 hrs.) | (72 hrs.) |
| 1 | Mn 10 | 74.4 | 62 | 65.3 |
| 2 | Mn 50 | 68 | 69.7 | 69.4 |
| 3 | Mn 80 | 71.7 | 70.3 | 68 |
| 4 | Mn 100 | 71.2 | 85.3 | 73.5 |
| 5 | Mn 150 | 78.2 | 84.1 | 78.2 |
| 6 | Cn 10 | 82.5 | 59.9 | 65.5 |
| 7 | Cn 50 | 74.4 | 70.5 | 70 |
| 8 | Cn 80 | 78.2 | 70.5 | 70 |
| 9 | Cn 100 | 86.3 | 72.6 | 79 |
| 10 | Cn 150 | 84 | 75.5 | 79.8 |
| 11 | P 100 | 76.9 | 80.7 | 79.6 |
| 12 | P 150 | 77.3 | 67.8 | 87.8 |

### Example 3

### Anti-Leishmanial Effects In Vivo of Green Synthesis of Silver Nanoparticles

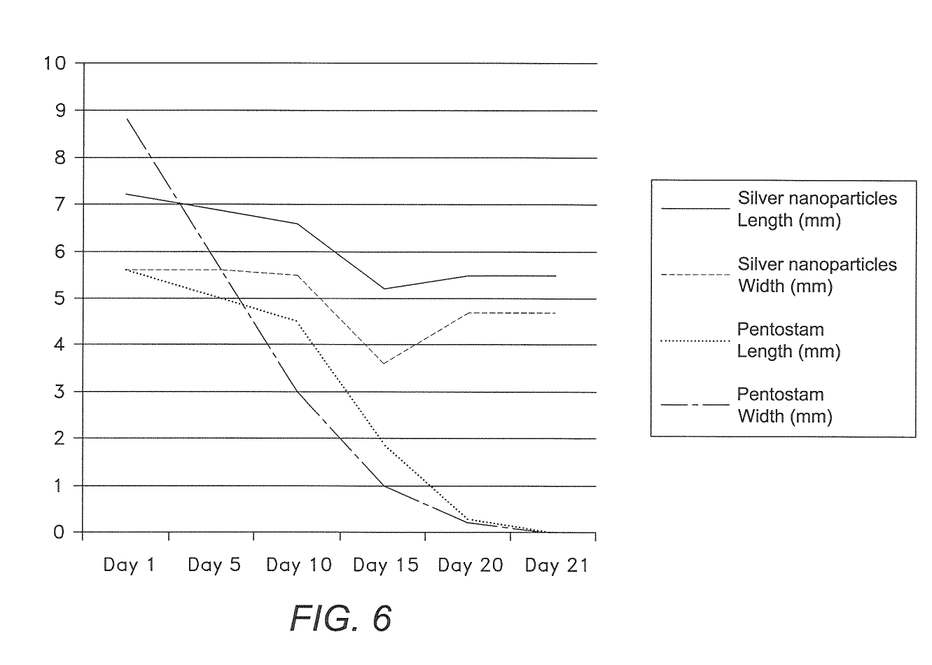

The balb/c mice were collected from the local market. They were injected with the Leishmania major parasite cultured in the lab. After 1 month, the infection on the skin appeared. Different treatments were applied to the lesions superficially for 21 days until the skin lesions healed completely. The experimental trial consisted of 2 groups and each group had 10 balb/c mice. The first treatment group was injected with 100 μl of Pentostam every day. The second treatment group was treated with silver nanoparticles synthesized by the gum extract of the plant Commiphara myrrha (Myrrh) in accordance with the method described in Example 1. The nanoparticles and the aqueous myrrh suspension in which the nanoparticles were formed were applied to the lesion of the balb/c mice superficially or externally on the lesion with the help of the dropper. The amount of the myrrh silver nanoparticle applied superficially was 2 ml-3 ml with the help of a dropper; this amount was enough to cover the wound completely. This treatment was given to the mice every day for 21 days until the lesion completely healed. FIG. 6 shows the length and width of the lesion in millimeters in the two groups of silver nanoparticles and Pentostam. Table 2 shows the decreasing size of the lesion and that it completely disappeared in 21 days when treated with silver nanoparticles.

TABLE 2
The Size of Lesion (mm) in the Two Treatment Groups

| | |
|---|---|
| Silver nanoparticles | Pentostam |

| | Length | Width | Length | Width |
|---|---|---|---|---|
| Time | (mm) | (mm) | (mm) | (mm) |
| Day 1 | 8.8 | 5.6 | 7.2 | 5.6 |
| Day 5 | 5.7 | 5 | 6.9 | 5.6 |
| Day 10 | 3 | 4.5 | 6.6 | 5.5 |
| Day 15 | 1 | 1.9 | 5.2 | 3.6 |
| Day 20 | 0.2 | 0.3 | 5.5 | 4.7 |
| Day 21 | 0 | 0 | 5.5 | 4.7 |

### Example 4

### Anti-Leishmanial Effects In Vivo of Green Synthesis of Silver Nanoparticles

Similar to the previous Example 3, balb/c mice were collected from the local market. The mice were injected with the Leishmania major parasite cultured in the lab. After 1 month, the infection on the skin appeared. Different treatments were applied to the lesions superficially for 21 days, as shown in Table 3. The experimental trial consisted of 4 groups and each group had 10 balb/c mice. The first treatment group was treated with myrrh silver nanoparticles, i.e., silver nanoparticles (mean size about 5 nm to about 50 nm) synthesized using the gum extract of the plant Commiphora myrrha (myrrh) as described in Example 1, and the aqueous myrrh suspension in which the silver nanoparticles were formed. The silver nanoparticles in aqueous myrrh suspension were applied to the lesion of the balb/c mice superficially or externally on the lesion with the help of the dropper. About 2 ml to about 3 ml of the myrrh silver nanoparticles were applied superficially for 21 days. The second treatment group was injected with 100 μl of Pentostam every day. The third treatment group was treated with commercially available silver nanoparticles (mean size about 5 nm to about 50 nm) in concentrations equal to that of the myrrh silver nanoparticles used for treating the first treatment group. The commercially available silver nanoparticles were applied superficially to the lesions in an amount of about 2 ml to about 3 ml with the help of a dropper for 21 days. The fourth treatment group was treated with just the gum extract of the plant Commiphora myrrha (Myrrh) without the silver nanoparticles in accordance with the method described in Example 1. Table 3 shows that the myrrh silver nanoparticles left in the aqueous myrrh suspension demonstrated better results than what was achieved by the commercially available silver nanoparticles and what was achieved by the aqueous myrrh extract without nanoparticles.

TABLE 3
The Size of Lesion (mm) in the Four Treatment Groups

| | | | | | | | Myrrh extract | |
|---|---|---|---|---|---|---|---|---|
| | Silver | | | | Commercial | | (without silver | |
| | nanoparticles | | Pentostam | | nanoparticles | | nanoparticles) | |
| | Length | Width | Length | Width | Length | Width | Length | |
| Time | (mm) | (mm) | (mm) | (mm) | (mm) | (mm) | (mm) | Width (mm) |
| Day 1 | 8.8 | 5.6 | 7.2 | 5.6 | 7.2 | 5.6 | 7.5 | 5.4 |
| Day 5 | 5.7 | 5 | 6.9 | 5.6 | 7 | 5.5 | 6.6 | 5.1 |
| Day 10 | 3 | 4.5 | 6.6 | 5.5 | 6.6 | 5.5 | 6 | 4.8 |
| Day 15 | 1 | 1.9 | 5.2 | 3.6 | 5.4 | 5.2 | 5.5 | 4.3 |
| Day 20 | 0.2 | 0.3 | 5.5 | 4.7 | 5.5 | 4.9 | 4.9 | 4 |
| Day 21 | 0 | 0 | 5.5 | 4.7 | 5.5 | 4.8 | 4.8 | 3.8 |

As illustrated in Table 3, the Leishmania lesions treated with silver nanoparticles prepared by the inventive method completely healed within the time period of 21 days. The silver nanoparticles together with the Myrrh extract/suspension were used directly on the lesions. In other words, the silver nanoparticles were not isolated from the aqueous Myrrh suspension prior to the topical treatment. The aqueous Myrrh with the synthesized silver nanoparticles is used as anti-leishmanial composition. However, when the Leishmania lesion was treated with commercial available silver nanoparticles (having a mean diameters of 5 nm to 50 nm similar to the Myrrh synthesized silver nanoparticles) only a partial healing was observed even after 21 days of treatment as shown on Table 3. It should be noted that the silver nanoparticles prepared from the inventive process were about 5-50 nm as shown in FIGS. 2 and 3. For comparison, Table 3 also provides the treatment effect of the commercial drug Pentostam as well as pure Myrrh extract (without the silver nanoparticles) on the Leishmania lesions, which surprisingly do not show complete recovery even after 21 days. As is clear to one of skill in the art from the comparative data of Myrrh extract with and without silver nanoparticles that the silver nanoparticles synthesized by the Myrrh extract is directly responsible for diminishing the lesions rather than the Myrrh extract or the commercially available silver nanoparticles.

It is to be understood that the present invention is not limited to the embodiments described above, but encompasses any and all embodiments within the scope of the following claims.

We claim:

1. A method of preparing metal nanoparticles from myrrh comprising:
preparing an extract of myrrh gum;
providing an aqueous solution including a metal salt; and
stirring the myrrh gum extract with the aqueous solution of the metal salt solution for about 15 to 20 minutes at a temperature of about 50° C. to about 100° C. to produce the metal nanoparticles.

2. The method of preparing metal nanoparticles from myrrh according to claim 1, wherein the extract and the aqueous solution are mixed at a temperature of about 60° C. 3 The method of preparing metal nanoparticles from myrrh according to claim 1, wherein the metal salt is silver nitrate (AgNO₃) and the metal nanoparticles are silver nanoparticles.

4. The method of preparing metal nanoparticles from myrrh according to claim 1, wherein the metal nanoparticles have a mean diameter in the range of from about 5 nm to about 50 nm.

5. The method of preparing metal nanoparticles from myrrh according to claim 1, wherein the metal nanoparticles are spherical, spheroidal, elongated spherical, rod-shaped, and/or faceted.

6. A method of treating epidermal lesions caused by Leishmaniasis comprising:
mixing an extract of myrrh with an aqueous solution of silver nitrate (AgNO₃) to form silver nanoparticles in aqueous myrrh suspension, the silver nanoparticles having a mean size of about 5 nm to about 50 nm; and
topically applying an effective amount of the silver nanoparticles in aqueous myrrh suspension to a patient in need thereof.

7. The method of treating epidermal lesions caused by leishmaniasis according to claim 6, wherein the leishmaniasis is caused by Leishmania major.

8. The method of treating epidermal lesions caused by leishmaniasis according to claim 6, wherein the lesions are subcutaneous epidermal lesions.

9. The method of treating epidermal lesions caused by leishmaniasis according to claim 6, wherein the metal nanoparticles are silver nanoparticles.

10. The method of treating epidermal lesions caused by leishmaniasis according to claim 9, wherein a concentration of the silver nanoparticles is between about 10⁻³ to 10⁻⁵ moles/L.

11. The method of treating epidermal lesions caused by leishmaniasis according to claim 10, wherein a concentration of the silver nanoparticles is between about 10⁻³ to 10⁻⁴ moles/L.
